(19) United States
(12) Patent Application Publication
(10) Pub. No.: US 2026/0276588 A1
(43) Pub. Date: Sep. 17, 2026

(54) Measuring the Net Magnetic Moment of a Geological Sample

(71) Applicant: Massachusetts Institute of Technology, Cambridge, MA (US)

(72) Inventors: Benjamin P. Weiss, Somerville, MA (US); Eduardo Andrade Lima, Belmont, MA (US)

(21) Appl. No.: 19/162,436

(22) PCT Filed: Apr. 11, 2024
(86) PCT No.: PCT/US2024/023988

§ 371 Date: Sep. 5, 2025

Related U.S. Application Data
(60) Provisional application No. 63/495,339, filed on Apr. 11, 2023.

(51) Int. Cl. G01N 27/72; G01N 33/24

(52) U.S. Cl. CPC G01N 27/72; G01N 33/24

(57) ABSTRACT

A method for determining the net magnetic moment of a sample having a magnetization distribution includes using a magnetic-field sensor to acquire a field map within a non-spherical area external to the sample, the field map comprising magnetic-field data that represents the magnetic field produced by the magnetization field of the sample; determining a plurality of representations of a magnetic field external to the sample, each of the representations being a spherical-harmonic multipole expansion that approximates the magnetic field, the expansion being a truncated expansion, selecting a final representation from among the representations based at least in part on the field map and on properties of the representations; and determining the net magnetic moment of the sample based at least in pail on the final representation.

### RELATED APPLICATIONS

This application claims the benefit of the Apr. 11, 2023 priority date of U.S. Provisional Application 63/495,339, the contents of which are incorporated herein by reference.

### STATEMENT REGARDING FEDERALLY SPONSORED RESEARCH OR DEVELOPMENT

This invention was made with government support under DMS1521765 and EAR2044806 awarded by the National Science Foundation and NNM16AA09C awarded by the National Aeronautics and Space Administration. The government has certain rights in the invention.

### BACKGROUND

During the process of formation, certain types of rocks start in a molten state and solidify as they cool. Many of these rocks have ferromagnetic constituents. When these rocks solidify in an ambient magnetic field, such as that of the Earth, the interaction between these constituents and the ambient magnetic field causes a statistical alignment of magnetic domains in ferromagnetic grains in the direction of this field. As a result, the rocks acquire a coherent, non-random magnetization distribution (hereafter “magnetization”). This magnetization persists long after the ambient magnetic field has changed or ceased. In many cases this magnetization is preserved over geologic time scales exceeding four billion years.

The fraction of the magnetization of a formed rock that is retained in the absence of any external magnetic field is called its “remanence,” or “remanent magnetization.” By measuring the rock's remanent magnetization, it is possible to infer what the ambient field must have been at the time of the rock's formation.

The foregoing process is not the only way that a rock can acquire remanence. For example, rocks that formed by compaction of sediments in lakes and oceans, i.e., “sedimentary rocks,” and rocks that experienced significant physical or chemical events after formation, such as high pressure, high temperature, and interaction with mineral-rich fluids, may also acquire a stable remanence that records the magnetic field at the time those processes occurred.

A difficulty that arises is that it is not usually possible to directly measure a rock's magnetization beyond a very thin surface layer, which is typically just several nanometers thick. Instead, one uses a magnetometer to measure the external magnetic field that is caused by the rock's magnetization. Based on this magnetometer measurement, one then solves an inverse problem: what particular magnetization would have resulted in the measured magnetic field?

This problem is somewhat difficult to solve for two reasons: non-uniqueness and noise contamination in the measurements.

As an initial matter, the solution to the inverse problem is not unique. There are, in fact, an infinite number of magnetization distributions that can produce the same observed external magnetic field.

Second, the solution itself is highly sensitive to noise. In fact, even small perturbations in the field data will result in disproportionately large changes in the solutions to the inverse problem.

Remanent magnetization is an emergent property of matter. It represents the aggregate behavior of many electrons. To obtain statistically meaningful constraints on ancient magnetic fields and to reduce the problem posed by a lack of uniqueness in the solution, it is sometimes preferable to measure a “net magnetic moment.” This net magnetic moment is essentially a volume integral of a magnetization distribution that exists within the rock. The volume of integration should be large enough to include a statistically significant number of magnetic grains. But it should not be so large that it encompasses portions of the rock that may have had a different geological history.

As a practical matter, it is often desirable to measure the external magnetic field of a rock from as close as possible to the rock. This is particularly true for rocks with complex magnetic histories, inhomogeneous composition, and/or weak magnetization. To achieve high spatial resolution and maximize the signal-to-noise ratio of the measurements, it is preferable to make a measurement in a plane that is parallel to a polished surface. This usually involves measuring one of the magnetic field's three components on a rectangular planar grid above the sample at close proximity. This results in a planar magnetic map of the rock sample's field associated with its remanence.

It is known to obtain an estimate of net magnetic moment for the entire volume of a sample based on certain approximations and mathematical relationships. However, these known methods are not applicable for magnetometers that measure a magnetic field along a plane at high spatial resolution.

### SUMMARY

The invention provides a way to infer the net magnetic moment of a geological sample based on a measurement of a map of the external magnetic field that is produced by that sample within a two-dimensional finite space proximate to the sample. The method includes carrying out truncated spherical-harmonic multipole expansions of the field evaluated in the rectangular measurement area based on the minimization of a cost function that results from comparing a modeled field map with the measured field map for plural estimates of an origin of the modeled field. The optimal truncation point of the multipole expansion is determined by weighing final costs and variation of the estimated moments across different truncation points.

In one aspect, the invention features a method for determining the net magnetic moment of a sample having a magnetization distribution. Such a method includes using a magnetic-field sensor to acquire a field map within a non-spherical area external to the sample. The field map includes magnetic-field data that represents the magnetic field produced by the magnetization distribution within the sample. The method continues with determining a plurality of representations of a magnetic field external to the sample. Each of the representations is a spherical-harmonic multipole expansion that approximates the magnetic field. This expansion is a truncated expansion. The method then continues with a step of selecting a final representation from among the representations based at least in part on the field map and on properties of the representations and determining the net magnetic moment of the sample based at least in part on the final representation.

In some practices, acquiring the field map includes acquiring the field map within a plane of finite extent. This plane is preferably parallel to a surface of the sample.

Other practices are those in which the step of determining a plurality of representations includes that of regularizing some of the representations to reduce sensitivity to numerical noise and experimental noise and those in which it includes estimating a different origin location for some of the representations.

Also among the practices are those in which each of the representations assumes a different number of combinations of magnetic dipoles at a fixed location.

Additional practices include those in which each of the representations is a weighted sum of terms, each of which includes a spherical harmonic function and a radial term. Each of the terms is a term in the expansion. These different representations differ in how many magnetic dipoles are used for the representation. In such practices, the method further includes, for each of the representations, selecting an origin, using regression on the field map to determine weights for the representation, using the weights, generating a modeled field, and using the field map and the modeled field, evaluating a cost function for the representation. This cost function characterizes a mismatch between the field map and the modeled field. The act of selecting the final representation includes selecting a representation based on the values of the cost function for all of the representations.

Still other practices include those in which determining a plurality of representations includes selecting a model degree that is associated with a representation. This model degree is related to how many magnetic dipoles are in the representation. Such practices continue with the steps of generating an initial estimate of an origin location for an origin for the representation, using the representation to obtain modeled field map, using the modeled field and the field map to evaluate a cost function, repeatedly using optimization algorithms to iteratively adjust the origin location to find a minimum of the cost function, the minimum being one of a global minimum and a local minimum, each of the adjustments beginning with a different initial estimate for the origin location, thereby obtaining a corresponding number of representations and improving a likelihood of convergence to the global minimum of the cost function, selecting, as the final representation, that representation for a given model degree that has the smallest cost, and selecting an overall final representation of the magnetic field across different model degrees based on a combination of the cost and properties of the representations.

Further practices include those in which each representation includes a superposition of polar terms. Among these are practices in which determining the plurality of representations includes truncating the superposition upon having determined that residuals are uncorrelated and at a level that is on average equal to a measurement noise level. In such practices, the residuals are derived from a mismatch between a modeled field and the field map. Also among these are practices in which each polar term has a degree and the step of determining the plurality of representations includes obtaining a plurality of estimates of the net magnetic moment and truncating the superposition upon having determined that the estimates of the net magnetic moment have begun to diverge as degree increases.

Also among these practices are those in which for each of the degrees, the method includes executing an inner loop, each iteration of which corresponds to a different estimate for an origin location.

In other practices of the method, determining the plurality of representations includes executing an inner loop, each iteration of which generates a different estimate of the net magnetic moment.

In still other practices of the method, determining the plurality of representations includes executing iterations of an inner loop. Each iteration of this inner loop includes the steps of generating a modeled field and determining a value of a cost function based on the modeled field and the field data. This cost function a measure of the mismatch between the field map and the modeled field.

In still other practices, determining the plurality of representations includes terminating execution of an inner loop upon determining that a cost function has attained a minimum. This cost function indicates a mismatch between the field data and a modeled field generated during the iteration.

In another aspect, the invention features an apparatus for determining the net magnetic moment of a sample having a magnetization distribution. Such an apparatus includes a magnetic-field sensor that has been configured to acquire a field map within a non-spherical area external to the sample. This field map includes magnetic-field data that represents the magnetic field produced by the magnetization distribution within the sample. The apparatus also includes certain measurement circuitry. This circuitry is configured to determine a plurality of representations of a magnetic field external to the sample. Each of the representations is a spherical-harmonic multipole expansion that approximates the magnetic field. The expansion is a truncated expansion. The circuitry is further configured to select a final representation from among the representations based at least in part on the field map and on properties of the representations and to then determine the net magnetic moment of the sample based at least in part on the final representation. Embodiments of the measurement circuitry include those in which the circuitry implements a digital computer, an analog computer, and/or application-specific integrated circuitry to implement the operations recited herein and to do so in a non-abstract manner and to do so with sufficient speed, accuracy, and reliability to prevent the operations from being practically carried out in the human mind via mental steps. In those embodiments in which the circuitry implements a digital computer, the digital computer is of the non-generic variety. Such a computer must be specially designed to interface correctly with a magnetic sensor and programmed to implement the desired operation. It has been discovered that such properties are not present in generic digital computers. In addition, the claims are to be construed to cover only non-abstract implementations. As used herein, “non-abstract” is the converse of “abstract” as that term has been defined in the Courts of the United States as of the filing of this application.

These and other features of the invention will be apparent from the following detailed description and the accompanying figures, in which:

### BRIEF DESCRIPTION OF THE FIGURES

FIG. 1 shows a schematic depiction of a magnetic microscope measuring the magnetic field due to a magnetized sample;

FIG. 2 shows a loop that regulates the number of polar terms in a multipole spherical-harmonic expansion of an approximation to the magnetic field of the sample shown in FIG. 1; and

FIG. 3 shows a loop that executed multiple times by the loop of FIG. 2 to evaluate cost functions associated with various approximations to the magnetic field of the sample shown in FIG. 1.

### DETAILED DESCRIPTION

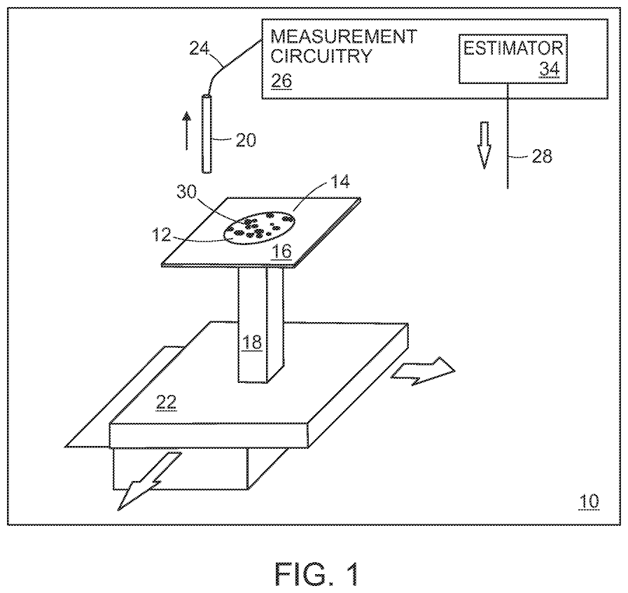

FIG. 1 shows a scanning magnetic microscope 10 for measuring a net magnetic moment of a magnetized sample 12 having a flat and polished top surface 14.

The sample 12 rests on a sample stage 16 that is raised by a pedestal 18 so that it is just under a magnetic-field sensor 20. The pedestal 18 sits on a movable scanning stage 22 that carries out a scanning motion. As a result, the magnetic-field sensor 20 is able to measure the external magnetic field due to the magnetized sample 12 at different points above the sample's top surface 14. The magnetic-field sensor 20 outputs a field map 24 to measurement circuitry 26, which ultimately outputs a measurement 24 of the sample's net magnetic moment 28.

Within the sample 12 or in its vicinity, it is convenient to define an origin 30. The origin 30 serves as the origin of a spherical coordinate system having a polar angle that is measured relative to an axis that is normal to the top surface 14. A reference sphere centered at the origin 30 surrounds the sample 12.

At each point within the sample 12, there exists a magnetization vector. This results in a magnetization distribution that causes the sample 12 to produce both internal and external magnetic fields, with the external field being a measurable quantity. The spatial distribution of this magnetization acts as the source for the sample's magnetic field. Thus, by measuring the sample's external magnetic field, it is possible to solve an inverse problem and recover the sample's magnetization distribution. This, in turn, provides insight into the ambient field that existed when the sample 12 acquired its magnetization.

In many cases, it is more advantageous to recover the sample's net magnetic moment 28 instead of its magnetization distribution. This net magnetic moment 28 is the integral of the magnetization distribution over the sample's volume and represents the sample's aggregate magnetization state. Based on the magnitude and direction of this net magnetic moment 28, it is possible to infer, through stepwise demagnetization and re-magnetization in a known laboratory field, the magnitude and direction of the ambient field at the time the magnetized sample 12 acquired its magnetization.

A convenient way to represent the sample's magnetic field is as a superposition of magnetic fields of point sources. Each of these magnetic fields arises from a different configuration of dipoles. This representation of the sample's magnetic field thus takes the form of an infinite series in which each term represents the field due to a particular configuration of magnetic poles. The first term would be the field due to three components of a dipole, the second would be the field due to five components of a quadrupole, the third would be a field due to the seven components of an octupole, and so on. Rather than referring to these terms by progressively more obscure Latin terms, it is convenient to identify them by an integer “degree.” Because these terms represent configurations of poles, they will be referred to herein as “polar terms” for convenience.

Each term in this infinite series of polar terms is represented by a spherical harmonic function combined with an inverse-power radial term, with this combination being referred to as a “solid harmonic.” For each multipole, the power of the radial term and the degree of the spherical harmonic function are fixed, with the order of the spherical harmonic function being associated with individual components of a multipole. This results in a finite series of spherical harmonics for each polar term. The finite series of spherical harmonics shall be referred to herein as the “inner series.” This inner series embodies just information about the angular dependence of the sample's magnetic field.

The infinite series of polar terms shall be referred to herein as the “outer series.” This outer series carries information about both the radial and angular dependences of the sample's magnetic field. This separation of the radial dependence conveniently arises because the partial differential equation that relates the magnetic field to its magnetization has separable solutions.

The foregoing representation of the magnetic field as an infinite series of finite series is often referred to as a “spherical-harmonic multipole expansion.” Assuming the location and radius of a reference sphere is chosen so that no sources of magnetization lie outside of the reference sphere, and all magnetic field measurements are taken on or outside the reference sphere, the dipole term in the multipole expansion is identical to the net magnetic moment of the sample and is the same irrespective of the size and location of the reference sphere.

To estimate a sample's net magnetic moment 28, it is necessary to measure the external magnetic field produced by the sample 12. In this case, the magnetic-field sensor 20 provides a set of measurements within a measurement plane that lies just above the top surface 14 of the sample 12. Because the magnetic field's amplitude diminishes rapidly with distance, it is useful to keep this distance as small as possible to maximize the field detected by the magnetic-field sensor 20. This also limits the size of the measurement region, as the magnetic field of the sample 12 falls below the noise floor of the magnetic-field sensor 20 past a certain distance.

The resulting set of measurements is a field map 24 that is provided to a net-magnetic-moment estimator 34 within the measurement circuitry 26.

The limited extent of the measurement plane comes at a cost. Although it results in a sensitive and spatially selective measurement of the sample 12, the measurement plane 18 spans only those polar angles defined by a narrow cone having a small cone angle. This differs from conventional measurements of bulk rock samples in which the magnetic field is measured on a cylindrical or spherical surface enveloping the sample and includes measurements from a significant portion of the reference sphere. This planar measurement region with limited range impairs the ability of conventional methods to accurately estimate the sample's net magnetic moment 28. In particular, when the field map 24 is measured over a plane instead of a sphere, the spherical harmonics that serve as an orthogonal basis for the relevant function space are no longer orthogonal and determination of one coefficient becomes dependent on the determination of all other coefficients in the expansion

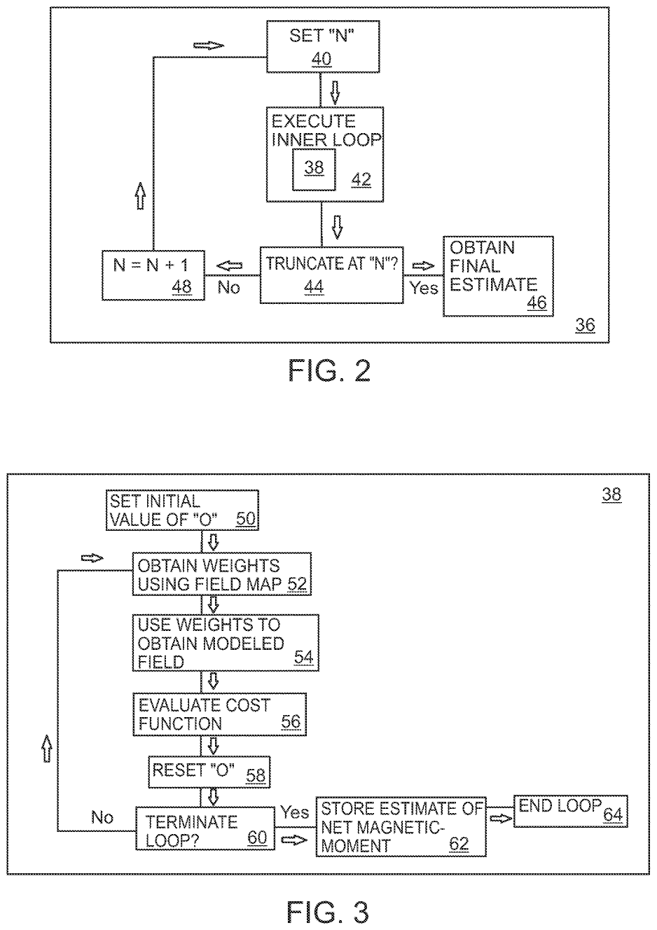

The magnetic-moment estimator 34 (hereafter referred to as the “estimator” for brevity) carries out the process discussed in connection with FIGS. 2 and 3 to output an estimate of the net magnetic moment 28 of the sample 12. This estimate involves using the spherical-harmonic multipole expansion. However, since one cannot very well carry infinite series through a computation, the outer summation must be truncated.

Once a multipole expansion is truncated, the location of the origin 30 of the expansion becomes critical for determining how well that expansion represents the measured data and how well it captures the net moment in the dipole term. Thus, it no longer holds true that the dipole term is invariant with respect to the location of the origin 30. In fact, some origin locations will perform much better than others in approximating the field and estimating the net magnetic moment.

The estimator 34 thus has two parameters for controlling its estimate of the net magnetic moment 28: the truncation point for the outer series and the location of the origin of the multipole expansion, with the radius of the reference sphere not being an independent quantity and instead being set as the distance between the origin and the measurement plane. One might expect that the more terms of the series one uses the more accurate the result will be. With this assumption, the choice of where to truncate comes down to determining a desired level of accuracy. The resulting truncated spherical harmonic multipole expansion approximates the magnetic field.

However, it turns out that there exist complications that make the optimal locations for truncation of infinite series more difficult to determine.

A difficulty arises because noise exists in the field map 24 that the magnetic-field sensor 20 provides to the estimator 34. In the course of evaluating the weights used in the inner summation, the estimator 34 carries out a least-squares regression using these noisy field data. As the number of terms in the inner series increases, the estimator 34 grows more sensitive to noise. It also attempts to use its additional degrees-of-freedom to approximate this noise. Thus, it is important to truncate the outer series early enough so as to minimize the impact of noise on the stability of the net magnetic moment measurement and to ensure that the estimator 34 approximates only the field data and not the noise.

Another difficulty arises because the origin 30 in FIG. 1 is not actually known. It must be estimated. A poor choice for this origin 30 has an insidious effect on the multipole expansion's rate of convergence.

The “magnetic” field arises from a distributed magnetic source, i.e., the magnetization distribution within the sample 12. However, this source is a vector quantity. It is not a nonnegative scalar quantity. This makes it difficult to identify a “center of magnetism” for the sample 12. In fact, the source term for the field external to the sample is actually the divergence of the magnetization distribution. As a result, any magnetization that has zero divergence would produce no external magnetic field. This makes it even more difficult to assign a center to a magnetization distribution.

It turns out that there exists an origin location that provides an optimal approximation of the measured field for a given truncation of the multipole expansion. Alternative choices for the origin location would require more multipole terms to be included in the truncated expansion to represent the field measurements with the same accuracy. The problem that arises from an incorrect origin location appears to be exacerbated by having the field map 24 come from a measurement planar region of such limited extent. An error in the origin's location, when coupled with the planar nature of the data, results in an ill-conditioned inverse problem. Such a problem requires the introduction of regularization to tame noise magnification in the solution. This regularization enables a trade-off between matching of the field data (and, indirectly, the accuracy with which the net magnetic moment can be measured) and the stability of the net magnetic moment measurements.

The process carried out by the estimator 34 generates candidate representations of the magnetic field, each of which approximates the magnetic field. It does so using an outer loop 36 and an inner loop 38, details of which are shown in FIGS. 2 and 3, respectively.

Referring now to FIG. 2, the estimator 34 begins the outer loop 36 by setting the degree of the polar term (step 40) to be that of the dipole term.

The outer loop 36 continues with several iterations of the inner loop 38 (step 42). The estimator 34 then inspects the outcome of the iterations of the inner loop 38 to see if an outer-loop truncation-condition is satisfied (step 44). This outcome takes the form of a distribution of residuals that is evaluated during the inner loop 38 combined with a measure of the stability of the solution.

If the outer-loop truncation-condition is satisfied, the estimator 34 ends the outer loop 36 and provides a final estimate of the net magnetic moment 15 (step 46). Otherwise, the estimator 34 increments the degree (step 48) and begins another iteration of the outer loop 36 (step 40).

The outer-loop truncation-condition is satisfied upon the occurrence of two events.

The first event arises if the distribution of residuals for the current model degree are nearly uncorrelated and at noise level or if estimates of the net magnetic moment 28 begin to diverge rapidly. In such cases, there exists an implication that enough polar terms have been added and therefore the outer loop 36 should be terminated.

The second event arises because the estimator 34 sets a maximum number of polar terms and halts the outer loop 36 when those polar terms required to yield an optimal estimate for the net moment have all been added to the multipole spherical-harmonic expansion.

During each iteration of the inner loop 38, the net-magnetic-moment estimator 34 generates a model of the field and compares that modeled field with the measured field data. This results in a value of a cost function, in a spatial distribution of the residuals, and in an estimate for the net moment. Thus, once the inner loop 38 stops executing, the outer loop 36 will have available to it a set of values of cost functions, distributions of residuals, and net moment estimates. This means that the outer loop 36 will be able to observe trends in the cost functions, residual distributions, and evolution of the solutions with increasing number of multipoles incorporated into the model.

If the trends reveal that the net magnetic moment estimates are stable and that the cost function is decreasing rapidly, the outer loop 36 increments the degree of the polar term and starts all over again. If the trends reveal that the stability of the solution is degrading while the residuals are decreasing at a slower pace and the residuals distribution is uncorrelated or nearly uncorrelated, it knows that it is time to stop the calculations and select the solution prior to the degradation in solution stability.

It is typically not advantageous to use more polar terms than needed. An excessive number of polar terms can promote numerical instability and magnify noise effects, not to mention increasing the computation time. In addition, an excessive number of terms raises the possibility of overfitting. As a result, it is not unusual for an estimate for the net magnetic moment 28 to converge to a steady value as the number of polar terms increases but to then diverge away from that value as more polar terms are used. The outer loop 36 is intended to locate a breakpoint at which convergence turns into divergence, thereby promoting a more accurate estimate of the net magnetic moment 28.

As shown in FIG. 3, the inner loop 38 begins by setting an initial value for the origin 12 (step 50).

The process of acquiring an initial estimate of the origin's location begins with providing the estimator 34 with an initial guess based on inspection of the field map 24 and on an estimate for the distance between the sample 12 and the magnetic-field sensor 16. In a preferred practice of the method, the inner loop 38 is carried out several times using different initial guesses. In most cases, at least thirty initial guesses appear to be adequate to provide satisfactory estimates by selecting the lowest cost function, thus nearly ensuring the likelihood that the global minimum in the cost function will be achieved using a given truncation of the spherical-harmonic multipole expansion.

The transverse coordinates of the origin typically lie in the vicinity of the location of maximum intensity of the total magnetic field. This information can thus be inferred from the observed magnetic field in the direction normal to the top surface 14. This phenomenon is largely independent of magnetization direction, thus making it particularly useful as an initial estimate for the origin's transverse coordinates.

The origin's vertical coordinate is somewhat more difficult to estimate. However, the distance between the top surface 14 of the sample 12 and the magnetic-field sensor 16 provides a lower limit for the vertical coordinate. In some cases, it is useful to incorporate the sample's thickness in the initial estimate of the origin's vertical coordinate.

The estimator 34 arranges the field data into a magnetic-data vector and provides the resulting magnetic-data vector to a non-linear optimizer. The estimator 34 also introduces random perturbations to the initial guess and then provides the resulting perturbed initial guess to the non-linear optimizer. In response, the non-linear optimizer generates its initial estimate of the origin's location. Examples of a non-linear optimizer are those that comprise circuitry for implementing one of: the Levenberg-Marquardt method, the trust-region-reflective method, and the simplex method.

Based on this estimate of the origin 12 and the measured field data, the estimator 34 obtains the weights for the solid harmonics (step 52). Using these weights, the estimator 34 then calculates a model field (step 54) and compares the modeled field with the field data to obtain a value of a cost function (step 56). Based on the value of this cost function and its evolution, the estimator 34 uses a nonlinear optimization method to produce a new value for the origin coordinates (step 58). After having done so, the estimator 34 determines whether a condition for terminating the optimization procedure has been met (step 60). A suitable condition is that of a minimum of the cost function having been reached or a maximum number of iterations having been exceeded, whichever comes first.

If so, the estimator 34 stores an estimate for the net magnetic moment 28 and uses a new origin estimate to begin another iteration of the inner loop 38. The inner loop 38 terminates when the prescribed number of optimizations with different starting points has been completed. Ultimately, this results in as many stored estimates as there are initial guesses. The solutions are then compared and the one with the lowest cost function value is passed on to the outer loop 38 as the net moment estimate obtained for a given truncation of the multipole expansion.

The step of obtaining the weights of the solid harmonics (step 52) includes using linear least-squares regression to solve a matrix equation to identify a column vector of unknown weights that, when operated on by a particular matrix, maps most closely to a column vector that represents the field map 24. For an outer series that truncates to leave behind only N polar terms, the dimensionality of the weight vector to be solved for is N²+2N. However, it is preferable that the system of equations be an overdetermined system to improve robustness of the solutions in the presence of measurement noise. This means that the field map 24 should have a dimensionality greater than N²+2N.

The matrix that operates on the weight vector comes from the terms of the truncated spherical harmonic expansion. Its column space has a dimensionality that is determined by the number of polar terms in the expansion and its row space has a dimensionality that is set by the number of measurements in the field map 24. This matrix may be modified to incorporate additional regularization to tame noise amplifications when the magnetic data arc noisy. One way of accomplishing this is via truncation of the singular value decomposition of such a matrix to limit its condition number.

The matrix then operates on the weight vector obtained by the linear regression to provide a modeled-field vector that represents the modeled field (step 54). This makes it possible to calculate residuals by taking the difference between the modeled-field vector and the field map 24. The norm of the resulting difference vector, suitably normalized by either the number of measurements in the field map 24 or the Euclidean norm of the measured field map, is the value of the cost function that corresponds to the estimate of the origin's location (step 58).

Having described the invention and a preferred embodiment thereof, what is new and secured by letters patent is:

1. A method for determining the net magnetic moment of a sample having a magnetization field, said method comprising:
using a magnetic-field sensor to acquire a field map within a non-spherical area external to said sample, said field map comprising magnetic-field data that represents the magnetic field produced by said magnetization field of said sample;
determining a plurality of representations of a magnetic field external to said sample, each of said representations being a spherical-harmonic multipole expansion that approximates said magnetic field, said expansion being a truncated expansion,
selecting a final representation from among said representations based at least in part on said field map and on properties of said representations; and
determining said net magnetic moment of said sample based at least in part on said final representation.

2. The method of claim 1, wherein acquiring said field map comprises acquiring said field map within a plane of finite extent, said plane being parallel to a surface of said sample.

3. The method of claim 1, wherein determining a plurality of representations includes regularizing some of said representations to reduce sensitivity to numerical noise and experimental noise.

4. The method of claim 1, wherein determining a plurality of representations comprises estimating a different origin location for some of said representations.

5. The method of claim 1, wherein each of said representations assumes a different number of combinations of magnetic dipoles at a fixed location.

6. The method of claim 1, wherein each of said representations is a weighted sum of terms, each of which comprises a spherical harmonic function and a radial term, wherein each of said terms is a term in said expansion, wherein different representations differ in how many magnetic dipoles are used for said representation, and wherein said method further comprises, for each of said representations, selecting an origin, using regression on said field map to determine weights for said representation, using said weights, generating a modeled field, using said field map and said modeled field, evaluating a cost function for said representation, wherein said cost function characterizes a mismatch between said field map and said modeled field, wherein selecting said final representation comprises selecting a representation based on the values of said cost function for all of said representations.

7. The method of claim 1, wherein determining a plurality of representations comprises selecting a model degree that is associated with a representation, said model degree being related to how many magnetic dipoles are in said representation, generating an initial estimate of an origin location for an origin for said representation, using said representation to obtain modeled field map, using said modeled field and said field map to evaluate a cost function, repeatedly using optimization algorithms to iteratively adjust said origin location to find a minimum of said cost function, said minimum being one of a global minimum and a local minimum, each of said adjustments beginning with a different initial estimate for said origin location, thereby obtaining a corresponding number of representations and improving a likelihood of convergence to said global minimum of said cost function, selecting, as said final representation, that representation for a given model degree that has the smallest cost, and selecting an overall final representation of said magnetic field across different model degrees based on a combination of said cost and properties of said representations.

8. The method of claim 1, wherein said representation comprises a superposition of polar terms, wherein determining said plurality of representations comprises truncating said superposition upon having determined that residuals are uncorrelated and at a level that is on average equal to a measurement noise level, wherein said residuals are derived from a mismatch between a modeled field and the field map.

9. The method of claim 1, wherein said representation comprises a superposition of polar terms, each polar term having a degree, wherein determining said plurality of representations comprises obtaining a plurality of estimates of said net magnetic moment and truncating said superposition upon having determined that said estimates of said net magnetic moment have begun to diverge as degree increases.

10. The method of claim 1, wherein said representation comprises a superposition of polar terms, each polar term having a degree, wherein determining said plurality of representations comprises, for each of said degrees, executing an inner loop, each iteration of which corresponds to a different estimate for an origin location.

11. The method of claim 1, wherein determining said plurality of representations comprises executing an inner loop, each iteration of which generates a different estimate of said net magnetic moment.

12. The method of claim 1, wherein determining said plurality of representations comprises executing iterations of an inner loop, each iteration comprising generating a modeled field and determining a value of a cost function based on said modeled field and said field data, said cost function being a measure of the mismatch between said field map and said modeled field.

13. The method of claim 1, wherein determining said plurality of representations comprises terminating execution of an inner loop upon determining that a cost function has attained a minimum, said cost function being indicative of a mismatch between said field data and a modeled field generated during said iteration.

14. An apparatus for determining the net magnetic moment of a sample having a magnetization field, said apparatus comprising:
a magnetic-field sensor configured to acquire a field map within a non-spherical area external to said sample, said field map comprising magnetic-field data that represents the magnetic field produced by said magnetization field of said sample and
measurement circuitry configured to determine a plurality of representations of a magnetic field external to said sample, each of said representations being a spherical-harmonic multipole expansion that approximates said magnetic field, said expansion being a truncated expansion, to select a final representation from among said representations based at least in part on said field map and on properties of said representations; and to determine said net magnetic moment of said sample based at least in part on said final representation.

15. The apparatus of claim 14, wherein said plurality of representations comprises a superposition of polar terms, wherein said measurement circuitry is configured to determine said plurality of representations by truncating said superposition upon having determined that residuals are uncorrelated and at a level that is on average equal to a measurement noise level, wherein said residuals are derived from a mismatch between a modeled field and the field map.

16. The apparatus of claim 14, wherein said representation comprises a superposition of polar terms, each polar term having a degree, wherein said measurement circuitry is configured to determine said plurality of representations by obtaining a plurality of estimates of said net magnetic moment and truncating said superposition upon having determined that said estimates of said net magnetic moment have begun to diverge as said degree increases.

17. The apparatus of claim 14, wherein each of said representations is a weighted sum of terms, each of which comprises a spherical harmonic function and a radial term, wherein each of said terms is a term in said expansion, wherein different representations differ in how many magnetic dipoles are used for said representation, and wherein, for each of said representations, said measurement circuitry is configured to carry out the steps of:
selecting an origin,
using regression on said field map to determine weights for said representation,
using said weights, generating a modeled field, and
using said field map and said modeled field, evaluating a cost function for said representation,
wherein said cost function characterizes a mismatch between said field map and said modeled field.

18. The apparatus of claim 14, wherein said measurement circuitry is configured to carry out the steps of:
selecting a model degree that is associated with a representation, said model degree being related to how many magnetic dipoles are in said representation,
generating an initial estimate of an origin location for an origin for said representation,
using said representation to obtain modeled field map,
using said modeled field and said field map to evaluate a cost function,
repeatedly using optimization algorithms to iteratively adjust said origin location to find a minimum of said cost function, said minimum being one of a global minimum and a local minimum, each of said adjustments beginning with a different initial estimate for said origin location, thereby obtaining a corresponding number of representations and improving a likelihood of convergence to said global minimum of said cost function,
selecting, as said final representation, that representation for a given model degree that has the smallest cost, and
selecting an overall final representation of said magnetic field across different model degrees based on a combination of said cost and properties of said representations.

19. The apparatus of claim 14, wherein said measurement circuitry is configured to determine said plurality of representations by executing iterations of an inner loop, each iteration comprising generating a modeled field and determining a measure of the mismatch between said field map and said modeled field.

20. The apparatus of claim 14, wherein said measurement circuitry is configured to determine said plurality of representations by terminating execution of an inner loop upon having determined that a mismatch between said field data and a modeled field generated during said iteration has attained a minimum.
